(19) United States
(12) Patent Application Publication
(10) Pub. No.: US 2025/0154452 A1
(43) Pub. Date: May 15, 2025

(54) PRODUCTION OF MEDIUM CHAIN LENGTH 3-HYDROXYACYL ACIDS

(71) Applicant: UT-Battelle, LLC, Oak Ridge, TN (US)

(72) Inventors: Adam M. Guss, Knoxville, TN (US); Walter Woodside, Knoxville, TN (US); Jay D. Huenemann, Knoxville, TN (US); Darren Parker, Knoxville, TN (US)

(21) Appl. No.: 18/941,567

(22) Filed: Nov. 8, 2024

Related U.S. Application Data
(60) Provisional application No. 63/547,874, filed on Nov. 9, 2023.

(51) Int. Cl. C12N 1/20; C12P 7/6409; C12R 1/40

(52) U.S. Cl. CPC C12N 1/20; C12P 7/6409; C12R 2001/40

(57) ABSTRACT

The present disclosure uses a combination of transcriptomics and genetics to demonstrate that P. putida PhaG is likely a 3-hydroxyacyl-ACP thiolase rather than a 3-hydroxyacyl-ACP:CoA transacylase. Deletion of two 3-hydroxyacyl CoA synthases results in the abolishment of PHAs as a product and leads to the accumulation of free medium chain length 3-hydroxyacyl acids as products into the culture supernatant under nitrogen starvation conditions. The present disclosure demonstrates a biological route to the production of 3-hydroxyacyl acids for use as industrial chemicals.

### CROSS REFERENCE TO RELATED APPLICATION

This application claims the benefit of priority from U.S. Provisional Application No. 63/547,874, filed on Nov. 9, 2023, the entire contents of which are incorporated herein by reference.

### STATEMENT REGARDING FEDERALLY SPONSORED RESEARCH OR DEVELOPMENT

This disclosure was made with government support under a research project supported by Prime Contract No. DE-AC05-00OR22725 awarded by the U.S. Department of Energy. The government has certain rights in this invention.

### INCORPORATION BY REFERENCE OF SEQUENCE LISTING

The Sequence Listing in an XML format, named as 43610_5204_01_SequenceListing of 30 KB, created on Nov. 6, 2024, and submitted to the United States Patent and Trademark Office via Patent Center, is incorporated herein by reference.

### BACKGROUND

Microbial carbon cycling in soil can convert decaying plant material into microbial biomass and then microbial necromass. Survival in variable environmental conditions necessitates that microbes generate stores of carbon, when conditions are favorable, to be drawn upon in the case of famine. Polyhydroxyalkanoates (PHAs) are one form of carbon storage that some soil microbes can generate and accumulate intracellularly. Some solely use the self-generated PHAs for energy while others can scavenge and break down PHAs extracellularly from necromass. PHA production begins with acetyl-CoA entering fatty acid biosynthesis, using Acyl Carrier Protein (ACP) as the carrier molecule. Once the carbon chain elongation reaches a length of approximately 8-10, PhaG is involved in pulling 3-hydroxyacyl units out of fatty acid biosynthesis for use in PHA biosynthesis. Previous biochemistry suggests that PhaG is a 3-hydroxyacyl-ACP:CoA transacylase, transferring the hydroxyacyl group from ACP to Coenzyme A. However, in-vivo studies in E. coli instead suggest that PhaG is a thiolase, hydrolyzing 3-hydroxyacyl-ACP to free Coenzyme A and free 3-hydroxy fatty acids.

Despite its use in metabolic engineering and PHA production, the reactivity of PhaG remains poorly understood as there are conflicting reports in the literature. Earlier reports, which included kinetic characterization, suggested PhaG possessing a CoASH-tranferase activity (3-hydroxyacyl-AcpP+CoASH↔3-hydroxyacyl-CoA+AcpP). Nevertheless, the transferase activity was only demonstrated in the reverse direction by monitoring the release of CoASH (Rehm et al, J Biol Chem. 1998 Sep. 11; 273 (37): 24044-51). By contrast, recent studies using E. coli strains engineered to overexpress the phaG gene led to extracellular accumulation of 3-hydroxydecanoic acid, consistent with a thioesterase activity (3-hydroxyacyl-AcpP+H₂O↔3-hydroxyacyl acid+AcpP) (Zheng et al, Antonie Van Leeuwenhoek 85, 93-101 (2004); Wang et al, Appl Environ Microbiol. 2012 January; 78 (2): 519-27).

Petroleum is a non-renewable resource used for nearly all modern plastic production. P. putida naturally produces and accumulates bioplastics called polyhydroxyalkanoates (PHAs), which is a polymer of 3-hydroxyacyl acids, when grown in nutrient limited conditions (e.g., nitrogen limitation). These bioplastics are useful for replacing a limited number of petroleum-based plastics and have the benefit of being biodegradable. However, chemical synthesis of PHAs allows for fine tuning of polymer properties, creating polymers with enhanced properties. Chemical synthesis requires free 3-hydroxyacyl acids as building blocks, but no known process exists for the biological production of these molecules.

### SUMMARY OF THE DISCLOSURE

The present disclosure seeks to replace petroleum-derived plastic monomers with sustainably produced ones. The present disclosure provides genetically engineered P. putida that produces and accumulates 3-hydroxyacyl acids, which are the building blocks needed for plastics that could be bio-based alternatives to petroleum-based ones.

One aspect of the present disclosure is directed to a genetically modified microbe, comprising a deletion of one or more nucleotides of at least one endogenous nucleic acid encoding an acyl (fatty acid) CoA ligase, wherein the genetically modified microbe displays an accumulation of medium chain length 3-hydroxyacyl acids under a nutrient starvation condition.

In some embodiments, the acyl CoA ligase is a medium chain length acyl CoA ligase. In some embodiments, the acyl CoA ligase is a 3-hydroxy acyl-CoA ligase.

In some embodiments, the acyl CoA ligase is selected from FadD1 or a homolog thereof, FadD2 or a homolog thereof, or AlkK or a homolog thereof.

In some embodiments, the acyl CoA ligase is FadD1 or a homolog thereof.

In some embodiments, the at least one endogenous nucleic acid comprises multiple endogenous nucleic acids, each encoding an acyl CoA ligase.

In some embodiments, the at least one endogenous nucleic acid comprises an endogenous nucleic acid encoding FadD1 or a homolog thereof, and an endogenous nucleic acid encoding AlkK or a homolog thereof.

In some embodiments, the at least one endogenous nucleic acid comprises an endogenous nucleic acid encoding FadD1 or a homolog thereof, an endogenous nucleic acid encoding FadD2 or a homolog thereof, and an endogenous nucleic acid encoding AlkK or a homolog thereof.

In some embodiments, the nucleic acid encoding the FadD1 or a homolog thereof comprises a nucleotide sequence having at least 90% sequence identity to the nucleotide sequence shown in SEQ ID NO: 1. In some embodiments, the FadD1 or the homolog thereof comprises an amino acid sequence having at least 90% sequence identity to the amino acid sequence shown in SEQ ID NO: 2.

In some embodiments, the nucleic acid encoding the AlkK or a homolog thereof comprises a nucleotide sequence having at least 90% sequence identity to the nucleotide sequence shown in SEQ ID NO: 3. In some embodiments, the AlkK or the homolog thereof comprises an amino acid sequence having at least 90% sequence identity to the amino acid sequence shown in SEQ ID NO: 4.

In some embodiments, the nucleic acid encoding the FadD2 or a homolog thereof comprises a nucleotide sequence having at least 90% sequence identity to the nucleotide sequence shown in SEQ ID NO: 5. In some embodiments, the FadD2 or the homolog thereof comprises an amino acid sequence having at least 90% sequence identity to the amino acid sequence shown in SEQ ID NO: 6.

In some embodiments, the deletion of one or more nucleotides of at least one endogenous nucleic acid encoding an acyl CoA ligase is a deletion of a portion of the at least one endogenous nucleic acid, and the resulting nucleic acid does not encode a functional acyl CoA ligase. In some embodiments, the deletion of one or more nucleotides of at least one endogenous nucleic acid encoding an acyl CoA ligase is a deletion of the full length of the at least one endogenous nucleic acid.

In some embodiments, the microbe is Pseudomonas. In some embodiments, the microbe is Pseudomonas putida.

In some embodiments, the medium chain length 3-hydroxyacyl acids comprises 6-12 carbon chain length compounds.

In some embodiments, the nutrient starvation condition comprises a nitrogen starvation condition.

In some embodiments, the genetically modified microbe is grown on an organic compound.

In some embodiments, the organic compound comprises a carbon source. In some embodiments, the carbon source comprises glucose, arabinose, xylose, glycerol, benzoate, acetate, p-coumaric acid, or terephthalate.

Another aspect of the disclosure is directed to a method of producing medium chain length 3-hydroxyacyl acids comprising growing a genetically modified microbe under a nutrient starvation condition, wherein the genetically modified microbe comprises a deletion of one or more nucleotides of at least one endogenous nucleic acid encoding an acyl (fatty acid) CoA ligase, and wherein the genetically modified microbe displays an accumulation of medium chain length 3-hydroxyacyl acids; and recovering medium chain length 3-hydroxyacyl acids produced by the microbe.

### BRIEF DESCRIPTION OF THE DRAWINGS

FIGS. 1A-1D. CoA ligase AlkK and FadD are required for PHA production. (A) RNA-seq analysis showing change in expression of CoA ligases when P. putida is grown on 3-hydroxydecanoate compared to acetate. (B, C) Plate reader growth assays of relevant strains grown on 3-hydroxy acids. Mean and standard error of three replicates shown for each strain. (D) GC-MS analysis showing carbon chain length composition of PHAs produced by the relevant strains.

FIGS. 2A-2B. 3-hydroxy acid accumulation in CoA ligase knockout strains. (A, B) HPLC analysis of culture supernatants from experiment shown in FIG. 1D. showing the detected 3-hydroxy acids.

### DETAILED DESCRIPTION

Native PHAs are not optimal for most plastic uses. PHAs chemically synthesized from a combination of medium chain 3-hydroxyacyl acids and short chain length 3-hydroxyacyl acids can overcome many limitations of the natively produced PHA variants. While heterologous microbial expression systems have demonstrated short chain 3-hydroxyacyl acid production, currently there are no existing methods for the production of medium chain 3-hydroxyacyl acids at high yield and titer. As P. putida has already been engineered to utilize a wide variety of sustainable and waste feedstocks (lignocellulosic sugars, aromatics derived from lignin, deconstructed plastics), manufacturing these bioplastic precursors could be done sustainably.

The present inventors believed that free 3-hydroxyacyl acids could be intermediates in the PHA production pathway, despite the fact that the pathway proposed in the literature does not contain this intermediate, and that CoA ligases may activate the 3-hydroxyacyl acids for PHA biosynthesis as well as for growth on 3-hydroxyacyl acids. The present disclosure identifies herein CoA ligase genes that are upregulated when P. putida is grown with medium chain 3-hydroxy acid as the sole carbon source. The present disclosure also provides herein a genetically modified bacterial strain incapable of utilizing medium-chain 3 hydroxy acids by deleting at least one CoA ligase gene from the chromosome. The present disclosure further demonstrates that the genetically modified bacterial strain accumulates medium chain length 3-hydroxyacyl acids when grown under nitrogen limited conditions instead of producing PHAs and provides a method of producing medium chain length 3-hydroxyacyl acids.

Unless defined otherwise, all technical and scientific terms used herein have the same meaning as commonly understood by one of ordinary skill in the art to which this invention belongs.

As used herein, the term “about” refers to an approximately +/−10% variation from a given value.

The term “microbe” as used herein, refers to an organism of microscopic size, which may exist in its single-celled form or as a colony of cells. In some embodiments, the microbe is a bacterium. In some embodiments, the microbe is Pseudomonas. In some embodiments, the microbe is Pseudomonas putida.

The term “genetically modified” or “genetically engineered” as used herein, refers to a microbe comprising a manipulated genome or nucleic acids. In some embodiments, the manipulation is a deletion of one or more nucleotides of at least one endogenous nucleic acid.

In some embodiments, the manipulation is a deletion of 1-10 nucleotides of at least one endogenous nucleic acid. In some embodiments, the manipulation is a deletion of at least 11 and up to 100 nucleotides of at least one endogenous nucleic acid. In some embodiments, the manipulation is a deletion of at least 100 nucleotides of at least one endogenous nucleic acid. In some embodiments, the manipulation is a deletion of at least 200 nucleotides of at least one endogenous nucleic acid. In some embodiments, the manipulation is the deletion of at least 300 nucleotides of at least one endogenous nucleic acid. In some embodiments, the manipulation is a deletion of the full length of the at least one endogenous nucleic acid.

A deletion of one or more nucleotides of an endogenous nucleic acid in a microbe can be accomplished by using any of the molecular engineering techniques known in the art. In some embodiments, an endogenous nucleic acid is deleted using kanamycin selection and sucrose counter selection method, as exemplified in the Examples hereinbelow.

In some embodiments, a deletion of one or more nucleotides of an endogenous nucleic acid leads to disruption of the activity of an enzyme encoded by the nucleic acid, so there is substantially no functional enzymatic activity of the encoded protein.

In some embodiments, the endogenous nucleic acid encodes an acyl (fatty acid) CoA ligase. In some embodiments, acyl CoA ligase catalyzes the activation of carboxylic acids via a two-step reaction of adenylation followed by thioesterification. In some embodiments, the acyl CoA ligase is a medium chain length acyl CoA ligase. In some embodiments, the acyl CoA ligase is a 3-hydroxy acyl-CoA ligase.

In this disclosure, the medium chain length 3-hydroxyacyl acids refer to 6-12 carbon chain length compounds. In some embodiments, the medium chain length 3-hydroxyacyl acids comprise 6 carbon chain length compounds. In some embodiments, the medium chain length 3-hydroxyacyl acids comprise 7 carbon chain length compounds. In some embodiments, the medium chain length 3-hydroxyacyl acids comprise 8 carbon chain length compounds. In some embodiments, the medium chain length 3-hydroxyacyl acids comprise 9 carbon chain length compounds. In some embodiments, the medium chain length 3-hydroxyacyl acids comprise 10 carbon chain length compounds. In some embodiments, the medium chain length 3-hydroxyacyl acids comprise 11 carbon chain length compounds. In some embodiments, the medium chain length 3-hydroxyacyl acids comprise 12 carbon chain length compounds. In some embodiments, the medium chain length 3-hydroxyacyl acids comprise a combination of compounds of different carbon chain lengths, from 6 carbon chain length up to 12 carbon chain length. In some embodiments, the medium chain length 3-hydroxyacyl acids comprises 8-12 carbon chain length compounds. In some embodiments, the medium chain length 3-hydroxyacyl acids comprises 10-12 carbon chain length compounds. In some embodiments, the medium chain length 3-hydroxyacyl acids comprises 11-12 carbon chain length compounds.

In some embodiments, the genetically modified microbe accumulates medium chain length 3-hydroxyacyl acids in a culture medium when grown under nutrient starvation conditions.

The term “nutrient starvation conditions”, as used herein, refers to conditions where a microbe is deliberately deprived of essential nutrients, causing them to enter a state of limited growth due to the lack of necessary building blocks for cell division and metabolism, often leading to physiological changes aimed at survival in the absence of readily available nutrients.

In some embodiments, a nutrient starvation condition comprises a nitrogen starvation condition. A “nitrogen starvation condition” as used herein refers to a growth condition in which the concentration of nitrogen is sufficiently low so as to become depleted while carbon source is still available, thus preventing further cell growth. As a normal nitrogen supply, microbes can be grown in tryptone-containing medium, such as LB (Miller) broth (10 g/L tryptone, 5 g/L yeast extract, and 10 g/L NaCl) conventionally used in the field. Other examples of normal nitrogen supply include a carbon source such as 25 mM p-coumaric acid (p-CA) and 10 mM (NH₄)₂SO₄. In contrast to such regular nitrogen source, an example of limited nitrogen source is a culture medium containing a carbon source such as 25 mM p-coumaric acid (p-CA) and 2 mM (NH₄)₂SO₄. Other examples of limited nitrogen source include a culture medium containing a carbon source such as 25 mM p-coumaric acid (p-CA) and 4 mM NaNO₃, or any condition in which the nitrogen source is depleted before the carbon source.

In some embodiments, the genetically modified microbe is grown on an organic compound as a carbon source. In some embodiments, the organic compound is selected from glucose, arabinose, xylose, glycerol, benzoate, acetate, p-coumaric acid, or terephthalate, or a combination thereof.

In some embodiments, the genetically modified microbe is grown on feedstock, for example, cellulosic sugars (e.g., glucose, xylose, arabinose), lignin-related aromatics (e.g., p-coumarate, ferulate), deconstructed plastics (e.g., terephthalate), organic acids (e.g., acetate), glycerol, or a combination thereof. As such, the genetically modified microbe disclosed herein can convert sustainable feedstocks and waste feedstocks into chemical precursors for next generation plastics.

In accordance with this disclosure, the genetically modified microbe accumulates medium chain length 3-hydroxyacyl acids in a culture medium at a high yield and titer when grown under nutrient starvation conditions. Unless specified, the term “yield” and “titer” refers to the amount and concentration of all medium chain length 3-hydroxyacyl acids. In some embodiments, the medium chain length 3-hydroxyacyl acids are accumulated at a titer of at least 0.2 g/L. In some embodiments, the medium chain length 3-hydroxyacyl acids are accumulated at a titer of at least 0.5 g/L. In some embodiments, the medium chain length 3-hydroxyacyl acids are accumulated at a titer greater than 1 g/L. In some embodiments, the medium chain length 3-hydroxyacyl acids are accumulated at a titer of at least 1.2 g/L. In some embodiments, the medium chain length 3-hydroxyacyl acids are accumulated at a titer of at least 1.5 g/L. In some embodiments, the medium chain length 3-hydroxyacyl acids are accumulated at a titer of at least 2.0 g/L. In some embodiments, the medium chain length 3-hydroxyacyl acids are accumulated at a titer of at least 2.5 g/L. In some embodiments, the medium chain length 3-hydroxyacyl acids are accumulated at a titer of at least 3.0 g/L.

In some embodiments, the acyl CoA ligase is selected from FadD1 or a homolog thereof, FadD2 or a homolog thereof, or AlkK or a homolog thereof.

In some embodiments, the acyl CoA ligase is FadD1 or a homolog thereof. In some embodiments, the at least one endogenous nucleic acid comprises multiple endogenous nucleic acids, each encoding an acyl CoA ligase. In some embodiments, the at least one endogenous nucleic acid comprises an endogenous nucleic acid encoding FadD1 or a homolog thereof, and an endogenous nucleic acid encoding AlkK or a homolog thereof.

In some embodiments, the term “homologous” refers to nucleic acids or polypeptides that are highly related at the level of nucleotide or amino acid sequence. Nucleic acids or polypeptides that are homologous to each other are termed “homologs”. A homolog of a specified gene generally comprises a nucleotide sequence that has a high degree of homology, e.g., sequence identity (at least 60%, 65%, 66%, 68%, 70%, 75%, 80%, 86%, 88%, 90%, 92%, 95%, 97% or 99%), to the sequence of the specified gene. A “homolog” of a specified gene generally also means that the function is highly related or equivalent to the function of the specified gene (e.g., encoding a protein having the same or similar enzymatic activity). A homolog of a specified protein generally comprises an amino acid sequence that has a high degree of homology, e.g., sequence identity (at least 60%, 65%, 66%, 68%, 70%, 75%, 80%, 86%, 88%, 90%, 92%, 95%, 97% or 99%), to the sequence of the specified protein. A “homolog” of a specified protein generally also means that the function is highly related or equivalent to the function of the specified protein (e.g., having the same or similar enzymatic activity). Homologs of a given gene and corresponding or equivalent positions in the homologous genes or proteins can be determined by sequence alignment programs, e.g., including but not limited to, NCBI BLAST, ClustalW, DIAMOND, CS-BLAST, and MAFFT.

As used herein, the term “nucleic acid” has its general meaning in the art and refers to a coding or non-coding nucleic sequence. Nucleic acids include DNA (deoxyribonucleic acid) and RNA (ribonucleic acid) nucleic acids.

In some embodiments, a nucleic sequence encoding FadD1 has a nucleotide sequence as laid out in SEQ ID NO: 1. In some embodiments, a nucleic sequence encoding a homolog of FadD1 has at least 85% sequence identity to the nucleotide sequence shown in SEQ ID NO: 1. In some embodiments, a nucleic sequence encoding a homolog of FadD1 has at least 90% sequence identity to the nucleotide sequence shown in SEQ ID NO: 1. In some embodiments, a nucleic sequence encoding a homolog of FadD1 has at least 91% sequence identity to the nucleotide sequence shown in SEQ ID NO: 1. In some embodiments, a nucleic sequence encoding a homolog of FadD1 has at least 92% sequence identity to the nucleotide sequence shown in SEQ ID NO: 1. In some embodiments, a nucleic sequence encoding a homolog of FadD1 has at least 93% sequence identity to the nucleotide sequence shown in SEQ ID NO: 1. In some embodiments, a nucleic sequence encoding a homolog of FadD1 has at least 94% sequence identity to the nucleotide sequence shown in SEQ ID NO: 1. In some embodiments, a nucleic sequence encoding a homolog of FadD1 has at least 95% sequence identity to the nucleotide sequence shown in SEQ ID NO: 1. In some embodiments, a nucleic sequence encoding a homolog of FadD1 has at least 96% sequence identity to the nucleotide sequence shown in SEQ ID NO: 1. In some embodiments, a nucleic sequence encoding a homolog of FadD1 has at least 97% sequence identity to the nucleotide sequence shown in SEQ ID NO: 1. In some embodiments, a nucleic sequence encoding a homolog of FadD1 has at least 98% sequence identity to the nucleotide sequence shown in SEQ ID NO: 1. In some embodiments, a nucleic sequence encoding a homolog of FadD1 has at least 99% sequence identity to the nucleotide sequence shown in SEQ ID NO: 1.

In some embodiments, SEQ ID NO: 1 is as shown below.

ATGATCGAAAATTTTTGGAAGGATAAGTACCCAGCCGGGATTACGGCG
GAAATCAATCCTGACGAATTCCCCAATATCCAGGCAGTACTCAAGCAATC
CTGCCAACGCTTTGCCGACAAACCGGCCTTTAGCAACCTGGGCAAGACTA
TCACTTATGGCGAGTTGTATGCGTTGTCGGGGGCGTTTGCCGCCTGGCTG
CAGCAGCATACCGACCTCAAGCCGGGTGACCGCATTGCCGTGCAACTGCC
CAATGTCCTGCAATACCCGGTCGCGGTCTTCGGTGCCATGCGTGCCGGGC
TGATCGTGGTCAACACCAACCCGCTGTACACCGCGCGGGAGATGGAACAC
CAGTTCAACGACTCGGGTGCCAAGGCCCTGGTGTGCCTGGCCAACATGGC
CCACCTGGCGGAAAAAGTGGTGCCCAAAACCCAGGTCAGGCACGTTATCG
TCACTGAAGTTGCCGACCTGCTGCCACCACTCAAGCGCCTGCTGATCAAC
AGCGTGATCAAGTACGTGAAGAAGATGGTGCCGGCCTACAACCTGCCGCA
GGCCGTGCGTTTCAATGACGCCCTGGCGCTGGGCAAGGGCCAGCCCGTGA
CCGAAGCCAACCCGCAGGCCAACGACGTGGCGGTGCTGCAGTACACCGGC
GGTACCACCGGTGTGGCCAAAGGCGCCATGCTGACCCACCGCAACCTGGT
GGCCAACATGCTGCAGTGCCGTGCACTGATGGGCTCCAACCTGCACGAAG
GCTGCGAAATCCTCATCACCCCGCTGCCGCTGTACCACATCTATGCGTTT
ACCTTCCATTGCATGGCGATGATGCTGATCGGCAACCACAACGTGCTGAT
CAGCAACCCGCGTGACTTGCCGGCGATGGTCAAGGAACTGGGCAAGTGGA
AGTTCAGCGGCTTTGTGGGCCTCAACACCCTGTTCGTTGCCCTGTGCAAC
AACGAGGCGTTCCGAGCCCTGGATTTCTCGGCGCTGAAAATCACCCTGTC
GGGCGGTATGGCCTTGCAGCTAAGCGTGGCCGAGCGCTGGAAGGCCGTTA
CCGGTTGCGCCATCTGCGAAGGCTACGGCATGACCGAAACCAGCCCGGTG
GCGGCGGTGAACCCCTCGGAAGCGAACCAGGTGGGCACCATCGGTATTCC
GGTGCCGTCGACCCTGTGCAAGGTCATCGACGACGCCGGCAATGAGTTGC
CGCTTGGCGAAGTGGGCGAGCTGTGTGTCAAGGGCCCGCAGGTGATGAAG
GGCTACTGGCAGCGTGAAGACGCCACGGCCGAGATTCTCGACAGCGAAGG
CTGGCTGAAGACCGGCGACATCGCCGTGATCCAGGCGGACGGCTACATGC
GCATCGTCGACCGCAAGAAAGACATGATCCTGGTCTCGGGCTTCAACGTA
TACCCCAACGAGCTGGAAGACGTGTTGGCGGCCCTGCCGGGCGTGCTGCA
GTGCGCAGCCATCGGTGTGCCGGACGAGAAGTCGGGTGAAGTGATCAAGG
TCTTCATCGTGGTCAAGCCGGGCATGACCGTGACCAAGGAGCAGGTGATG
GAGCACATGCGTGCCAACGTTACCGGCTACAAGGTACCGCGCCACATCGA
GTTCCGCGATTCGCTGCCGACCACCAACGTGGGCAAGATCCTGCGCCGCG
AACTGCGTGATGAAGAGCTCAAGAAGCAAGGCTTGAAGAAGATCGCCTGA

In some embodiments, FadD1 has an amino acid sequence as laid out in SEQ ID NO: 2. In some embodiments, a homolog of FadD1 has at least 85% sequence identity to the amino acid sequence shown in SEQ ID NO: 2. In some embodiments, a homolog of FadD1 has at least 90% sequence identity to the amino acid sequence shown in SEQ ID NO: 2. In some embodiments, a homolog of FadD1 has at least 91% sequence identity to the amino acid sequence shown in SEQ ID NO: 2. In some embodiments, a homolog of FadD1 has at least 92% sequence identity to the amino acid sequence shown in SEQ ID NO: 2. In some embodiments, a homolog of FadD1 has at least 93% sequence identity to the amino acid sequence shown in SEQ ID NO: 2. In some embodiments, a homolog of FadDlhas at least 94% sequence identity to the amino acid sequence shown in SEQ ID NO: 2. In some embodiments, a homolog of FadD1 has at least 95% sequence identity to the amino acid sequence shown in SEQ ID NO: 2. In some embodiments, a homolog of FadD1 has at least 96% sequence identity to the amino acid sequence shown in SEQ ID NO: 2. In some embodiments, a homolog of FadD1 has at least 97% sequence identity to the amino acid sequence shown in SEQ ID NO: 2. In some embodiments, a homolog of FadD1 has at least 98% sequence identity to the amino acid sequence shown in SEQ ID NO: 2. In some embodiments, a homolog of FadD1 has at least 99% sequence identity to the amino acid sequence shown in SEQ ID NO: 2.

In some embodiments, SEQ ID NO: 2 is as shown below.

MIENFWKDKYPAGITAEINPDEFPNIQAVLKQSCQRFADKPAFSNLGKT
ITYGELYALSGAFAAWLQQHTDLKPGDRIAVQLPNVLQYPVAVFGAMRA
GLIVVNTNPLYTAREMEHQFNDSGAKALVCLANMAHLAEKVVPKTQVRH
VIVTEVADLLPPLKRLLINSVIKYVKKMVPAYNLPQAVRENDALALGKG
QPVTEANPQANDVAVLQYTGGTTGVAKGAMLTHRNLVANMLQCRALMGS
NLHEGCEILITPLPLYHIYAFTFHCMAMMLIGNHNVLISNPRDLPAMVK
ELGKWKFSGFVGLNTLFVALCNNEAFRALDFSALKITLSGGMALQLSVA
ERWKAVTGCAICEGYGMTETSPVAAVNPSEANQVGTIGIPVPSTLCKVI
DDAGNELPLGEVGELCVKGPQVMKGYWQREDATAEILDSEGWLKTGDIA
VIQADGYMRIVDRKKDMILVSGFNVYPNELEDVLAALPGVLQCAAIGVP
DEKSGEVIKVFIVVKPGMTVTKEQVMEHMRANVTGYKVPRHIEFRDSLP
TTNVGKILRRELRDEELKKQGLKKIA

In some embodiments, a nucleic sequence encoding AlkK has a nucleotide sequence as laid out in SEQ ID NO: 3. In some embodiments, a nucleic sequence encoding a homolog of AlkK has at least 85% sequence identity to the nucleotide sequence shown in SEQ ID NO: 3. In some embodiments, a nucleic sequence encoding a homolog of AlkK has at least 90% sequence identity to the nucleotide sequence shown in SEQ ID NO: 3. In some embodiments, a nucleic sequence encoding a homolog of AlkK has at least 91% sequence identity to the nucleotide sequence shown in SEQ ID NO: 3. In some embodiments, a nucleic sequence encoding a homolog of AlkK has at least 92% sequence identity to the nucleotide sequence shown in SEQ ID NO: 3. In some embodiments, a nucleic sequence encoding a homolog of AlkK has at least 93% sequence identity to the nucleotide sequence shown in SEQ ID NO: 3. In some embodiments, a nucleic sequence encoding a homolog of AlkK has at least 94% sequence identity to the nucleotide sequence shown in SEQ ID NO: 3. In some embodiments, a nucleic sequence encoding a homolog of AlkK has at least 95% sequence identity to the nucleotide sequence shown in SEQ ID NO: 3. In some embodiments, a nucleic sequence encoding a homolog of AlkK has at least 96% sequence identity to the nucleotide sequence shown in SEQ ID NO: 3. In some embodiments, a nucleic sequence encoding a homolog of AlkK has at least 97% sequence identity to the nucleotide sequence shown in SEQ ID NO: 3. In some embodiments, a nucleic sequence encoding a homolog of AlkK has at least 98% sequence identity to the nucleotide sequence shown in SEQ ID NO: 3. In some embodiments, a nucleic sequence encoding a homolog of AlkK has at least 99% sequence identity to the nucleotide sequence shown in SEQ ID NO: 3.

In some embodiments, SEQ ID NO: 3 is as shown below.

ATGTTGCAGACACGCATCATCAAGCCCGCCGAGGGCGCCTATGCCTATC
CATTGCTGATCAAGCGCCTGCTGATGTCCGGCAGCCGCTATGAAAAGACC
CGGGAAATCGTCTACCGCGACCAGATGCGGCTGACGTATCCACAGCTCAA
CGAGCGCATTGCCCGCCTGGCCAACGTGCTGACCGAGGCCGGGGTCAAGG
CCGGTGACACCGTGGCGGTGATGGACTGGGACAGCCATCGCTACCTGGAA
TGCATGTTCGCCATCCCGATGATCGGCGCTGTGGTGCACACCATCAACGT
GCGCCTGTCGCCCGAGCAGATCCTCTACACCATGAACCATGCCGAAGACC
GCGTGGTGCTGGTCAACAGCGACTTCGTCGGCCTGTACCAGGCCATCGCC
GGGCAGCTGACCACTGTCGACAAGACCCTGCTACTGACCGATGGCCCGGA
CAAGACTGCCGAACTGCCCGGTCTGGTCGGCGAGTATGAGCAGCTGCTGG
CTGCTGCCAGCCCGCGCTACGACTTCCCGGATTTCGACGAGAATTCGGTG
GCCACTACCTTCTACACCACTGGCACCACCGGTAACCCCAAGGGCGTGTA
TTTCAGTCACCGCCAGCTGGTGCTGCACACCCTGGCCGAGGCCTCGGTCA
CCGGCAGTATCGACAGCGTGCGCCTGCTGGGCAGCAACGATGTGTACATG
CCCATCACCCCGATGTTCCACGTGCATGCCTGGGGCATCCCCTACGCTGC
CACCATGCTCGGCATGAAGCAGGTGTACCCAGGGCGCTACGAGCCGGACA
TGCTGGTCAAGCTTTGGCGTGAAGAGAAGGTCACTTTCTCCCACTGCGTG
CCGACCATCCTGCAGATGCTGCTCAACTGCCCGAACGCCCAGGGGCAGGA
CTTCGGCGGCTGGAAGATCATCATCGGCGGCAGCTCGCTCAACCGTTCGC
TGTACCAGGCCGCCCTGGCGCGCGGCATCCAGCTGACCGCCGCGTATGGC
ATGTCGGAAACCTGCCCGCTGATCTCCGCGGCACACCTGAACGATGAACT
GCAGGCCGGCAGCGAGGATGAGCGCGTCACTTACCGTATCAAGGCCGGTG
TGCCGGTGCCGTTGGTCGAAGCGGCCATCGTCGACGGCGAAGGCAACTTC
CTGCCCGCCGATGGTGAAACCCAGGGCGAGCTGGTACTGCGTGCGCCGTG
GCTGACCATGGGCTACTTCAAGGAGCCGGAGAAGAGCGAGGAGCTGTGGC
AGGGCGGCTGGCTGCACACCGGTGACGTCGCCACCCTCGACGGCATGGGC
TACATCGACATCCGCGACCGCATCAAGGATGTGATCAAGACCGGTGGCGA
GTGGGTTTCCTCGCTCGACCTGGAAGACCTGATCAGCCGCCACCCGGCCG
TGCGCGAAGTGGCGGTGGTGGGGGTGGCCGACCCGCAGTGGGGTGAGCGC
CCGTTTGCCCTGCTGGTGGCACGTGACGGCCACGATATCGACGCCAAGGC
GCTGAAGGAACACCTCAAGCCATTCGTCGAGCAAGGTCATATCAACAAGT
GGGCGATTCCAAGCCAGATCGCCCTTGTTACTGAAATTCCCAAGACCAGT
GTCGGCAAGCTCGACAAGAAACGCATTCGCCAGGACATCGTCCAGTGGCA
GGCCAGCAACAGCGCGTTCCTTTCCACGTTGTAA

In some embodiments, AlkK has an amino acid sequence as laid out in SEQ ID NO: 4. In some embodiments, a homolog of AlkK has at least 85% sequence identity to the amino acid sequence shown in SEQ ID NO: 4. In some embodiments, a homolog of AlkK has at least 90% sequence identity to the amino acid sequence shown in SEQ ID NO: 4. In some embodiments, a homolog of AlkK has at least 91% sequence identity to the amino acid sequence shown in SEQ ID NO: 4. In some embodiments, a homolog of AlkK has at least 92% sequence identity to the amino acid sequence shown in SEQ ID NO: 4. In some embodiments, a homolog of AlkK has at least 93% sequence identity to the amino acid sequence shown in SEQ ID NO: 4. In some embodiments, a homolog of AlkK has at least 94% sequence identity to the amino acid sequence shown in SEQ ID NO: 4. In some embodiments, a homolog of AlkK has at least 95% sequence identity to the amino acid sequence shown in SEQ ID NO: 4. In some embodiments, a homolog of AlkK has at least 96% sequence identity to the amino acid sequence shown in SEQ ID NO: 4. In some embodiments, a homolog of AlkK has at least 97% sequence identity to the amino acid sequence shown in SEQ ID NO: 4. In some embodiments, a homolog of AlkK has at least 98% sequence identity to the amino acid sequence shown in SEQ ID NO: 4. In some embodiments, a homolog of AlkK has at least 99% sequence identity to the amino acid sequence shown in SEQ ID NO: 4.

In some embodiments, SEQ ID NO: 4 is as shown below.

MLQTRIIKPAEGAYAYPLLIKRLLMSGSRYEKTREIVYRDQMRLTYPQL
NERIARLANVLTEAGVKAGDTVAVMDWDSHRYLECMFAIPMIGAVVHTI
NVRLSPEQILYTMNHAEDRVVLVNSDFVGLYQAIAGQLTTVDKTLLLTD
GPDKTAELPGLVGEYEQLLAAASPRYDFPDFDENSVATTFYTTGTTGNP
KGVYFSHRQLVLHTLAEASVTGSIDSVRLLGSNDVYMPITPMFHVHAWG
IPYAATMLGMKQVYPGRYEPDMLVKLWREEKVTFSHCVPTILQMLLNCP
NAQGQDFGGWKIIIGGSSLNRSLYQAALARGIQLTAAYGMSETCPLISA
AHLNDELQAGSEDERVTYRIKAGVPVPLVEAAIVDGEGNFLPADGETQG
ELVLRAPWLTMGYFKEPEKSEELWQGGWLHTGDVATLDGMGYIDIRDRI
KDVIKTGGEWVSSLDLEDLISRHPAVREVAVVGVADPQWGERPFALLVA
RDGHDIDAKALKEHLKPFVEQGHINKWAIPSQIALVTEIPKTSVGKLDK
KRIRQDIVQWQASNSAFLSTL

In some embodiments, a nucleic sequence encoding FadD2 has a nucleotide sequence as laid out in SEQ ID NO: 5. In some embodiments, a nucleic sequence encoding a homolog of FadD2 has at least 85% sequence identity to the nucleotide sequence shown in SEQ ID NO: 5. In some embodiments, a nucleic sequence encoding a homolog of FadD2 has at least 90% sequence identity to the nucleotide sequence shown in SEQ ID NO: 5. In some embodiments, a nucleic sequence encoding a homolog of FadD2 has at least 91% sequence identity to the nucleotide sequence shown in SEQ ID NO: 5. In some embodiments, a nucleic sequence encoding a homolog of FadD2 has at least 92% sequence identity to the nucleotide sequence shown in SEQ ID NO: 5. In some embodiments, a nucleic sequence encoding a homolog of FadD2 has at least 93% sequence identity to the nucleotide sequence shown in SEQ ID NO: 5. In some embodiments, a nucleic sequence encoding a homolog of FadD2 has at least 94% sequence identity to the nucleotide sequence shown in SEQ ID NO: 5. In some embodiments, a nucleic sequence encoding a homolog of FadD2 has at least 95% sequence identity to the nucleotide sequence shown in SEQ ID NO: 5. In some embodiments, a nucleic sequence encoding a homolog of FadD2 has at least 96% sequence identity to the nucleotide sequence shown in SEQ ID NO: 5. In some embodiments, a nucleic sequence encoding a homolog of FadD2 has at least 97% sequence identity to the nucleotide sequence shown in SEQ ID NO: 5. In some embodiments, a nucleic sequence encoding a homolog of FadD2 has at least 98% sequence identity to the nucleotide sequence shown in SEQ ID NO: 5. In some embodiments, a nucleic sequence encoding a homolog of FadD2 has at least 99% sequence identity to the nucleotide sequence shown in SEQ ID NO: 5.

In some embodiments, SEQ ID NO: 5 is as shown below.

ATGCAAGCCGACTTCTGGAATGACAAGCGCCCGGCAGGCGTGCCTTCC
ACCATCGACATCAATGCCTACGCCTCGGTCGTCGAGGTGTTCGAGCGCTC
CTGCAAGCGCTTTGCCGACCGCCCGGCGTTCAGCAACCTGGGCGTGACCC
TCAGCTACGCGGAACTGGAGCGCCATTCGGCAGCCTTCGCTGCCTGGTTG
CAGCAGCACACCGACCTCAAACCGGGTGAGCGCATCGCCGTACAGATGCC
CAATGTGCTGCAGTACCCCATCGCCGTCTTCGGTGCCATGCGCGCCGGGC
TGATCGTGGTCAATACCAACCCGCTGTACACCGAGCGCGAGATGCGCCAC
CAGTTCAAGGACAGTGGCGCGCGTGCGCTGGTGTACCTGAACATGTTCGG
CAAGCGCGTGCAGGAGGTGCTGCCCGATACCGGTATCGAATACCTGATCG
AGGCAAAGATGGGTGACCTGCTGCCGGCCGCCAAGGGCTGGCTGGTCAAC
ACCGTGGTCGACAAGTTGAAGAAGATGGTGCCGGCCTACCGGTTGCCCCA
GGCGGTGCCGTTCAAGCAGGTGCTGCGCGAGGGCCGCGGGCTGTCGCCCA
AACCGGTGTCGCTGAACCTCGATGACATCGCGGTGCTGCAGTACACCGGC
GGCACCACCGGCCTGGCCAAGGGCGCCATGCTCACCCATGGCAACCTGGT
GGCCAACATGCTGCAAGTGCTGGCCTGCTTCTCGCAGCACGGCCCCGATG
GGCAGAAGCTGCTGAAGGACGGCCAGGAAGTGATGATTGCGCCGCTGCCG
CTGTACCACATCTATGCCTTCACCGCGAACTGCATGTGCATGATGGTCAC
CGGCAACCATAATGTGCTGATTACCAACCCGCGGGATATTCCCGGCTTCA
TCAAGGAGCTGGGCAAGTGGCGTTTCTCTGCCTTGCTGGGCCTCAATACC
CTGTTTGTCGCACTGATGGACCACCCGGGATTCCGTCAGCTGGACTTCTC
GGCGCTGAAGGTCACCAACTCTGGTGGCACAGCGCTGGTCAAAGCCACCG
CCGAGCGCTGGGAAGACCTTACCGGGTGCCGCATCGTTGAAGGCTACGGC
CTGACAGAAACCTCGCCGGTGGCCAGCACTAACCCCTACGGCCAGCTGGC
GCGTCTGGGTACCGTGGGCATCCCCGTGGCGGGTACCGCGTTCAAGGTCA
TCGATGATGATGGCAACGAACTGCCGCTGGGCGAGCGGGGCGAGCTTTGC
ATCAAAGGCCCGCAGGTCATGAAGGGGTACTGGCAGCAGCCCGAGGCAAC
GGCCCAGGCACTGGATGCAGAAGGCTGGTTCAAGACCGGTGATATCGCGG
TGATCGACCCGGACGGCTTCACCCGAATTGTCGACCGCAAGAAGGACATG
ATCATCGTCTCGGGCTTCAACGTGTACCCCAACGAAATCGAGGATGTGGT
GATGGGGCATCCCAAGGTCGCCAACTGTGCGGCTATCGGCGTGCCGGACG
AGCGTTCCGGCGAGGCGGTCAAGCTGTTCGTGGTGCCGCGCGAGGGGGGG
CTGAGCGTTGATGAGCTGAAGGCCTACTGCAAGGCCAACTTCACCGGCTA
CAAAGTACCCAAGCACATCGTGCTGCGCGAATCGTTGCCCATGACGCCGG
TGGGCAAGATTTTGCGGCGGGAGTTGCGCGATATAGCGTGA

In some embodiments, FadD2 has an amino acid sequence as laid out in SEQ ID NO: 6. In some embodiments, a homolog of FadD2 has at least 85% sequence identity to the amino acid sequence shown in SEQ ID NO: 6. In some embodiments, a homolog of FadD2 has at least 90% sequence identity to the amino acid sequence shown in SEQ ID NO: 6. In some embodiments, a homolog of FadD2 has at least 91% sequence identity to the amino acid sequence shown in SEQ ID NO: 6. In some embodiments, a homolog of FadD2 has at least 92% sequence identity to the amino acid sequence shown in SEQ ID NO: 6. In some embodiments, a homolog of FadD2 has at least 93% sequence identity to the amino acid sequence shown in SEQ ID NO: 6. In some embodiments, a homolog of FadD2 has at least 94% sequence identity to the amino acid sequence shown in SEQ ID NO: 6. In some embodiments, a homolog of FadD2 has at least 95% sequence identity to the amino acid sequence shown in SEQ ID NO: 6. In some embodiments, a homolog of FadD2 has at least 96% sequence identity to the amino acid sequence shown in SEQ ID NO: 6. In some embodiments, a homolog of FadD2 has at least 97% sequence identity to the amino acid sequence shown in SEQ ID NO: 6. In some embodiments, a homolog of FadD2 has at least 98% sequence identity to the amino acid sequence shown in SEQ ID NO: 6. In some embodiments, a homolog of FadD2 has at least 99% sequence identity to the amino acid sequence shown in SEQ ID NO: 6.

In some embodiments, SEQ ID NO: 6 is as shown below.

MQADFWNDKRPAGVPSTIDINAYASVVEVFERSCKRFADRPAFSNLGVT
LSYAELERHSAAFAAWLQQHTDLKPGERIAVQMPNVLQYPIAVFGAMRA
GLIVVNTNPLYTEREMRHQFKDSGARALVYLNMFGKRVQEVLPDTGIEY
LIEAKMGDLLPAAKGWLVNTVVDKLKKMVPAYRLPQAVPFKQVLREGRG
LSPKPVSLNLDDIAVLQYTGGTTGLAKGAMLTHGNLVANMLQVLACFSQ
HGPDGQKLLKDGQEVMIAPLPLYHIYAFTANCMCMMVTGNHNVLITNPR
DIPGFIKELGKWRFSALLGLNTLFVALMDHPGFRQLDFSALKVTNSGGT
ALVKATAERWEDLTGCRIVEGYGLTETSPVASTNPYGQLARLGTVGIPV
AGTAFKVIDDDGNELPLGERGELCIKGPQVMKGYWQQPEATAQALDAEG
WFKTGDIAVIDPDGFTRIVDRKKDMIIVSGFNVYPNEIEDVVMGHPKVA
NCAAIGVPDERSGEAVKLFVVPREGGLSVDELKAYCKANFTGYKVPKHI
VLRESLPMTPVGKILRRELRDIA

In some embodiments, the deletion of one or more nucleotides of at least one endogenous nucleic acid encoding an acyl CoA ligase is a deletion of a portion of the at least one endogenous nucleic acid, and the resulting nucleic acid does not encode a functional acyl CoA ligase.

In some embodiments, the deletion of one or more nucleotides of at least one endogenous nucleic acid encoding an acyl CoA ligase is a deletion of the full length of the at least one endogenous nucleic acid.

In the present disclosure, three acyl CoA ligases have been identified as FadD1, AlkK and FadD2. The deletion of at least one nucleic acid encoding an acyl CoA ligase can be directed to any one of the three genes encoding FadD1, AlkK and FadD2, or any two of the three genes, or all three genes. In some embodiments, a deletion of one or more nucleotides is made in an endogenous gene encoding FadD1. In some embodiments, a deletion of one or more nucleotides is made in an endogenous gene encoding FadD1 and in an endogenous gene encoding AlkK. In some embodiments, a deletion of one or more nucleotides is made in all three genes, i.e., an endogenous gene encoding FadD1, an endogenous gene encoding FadD2, and an endogenous gene encoding AlkK.

In some embodiments, a method of producing medium chain length 3-hydroxyacyl acids comprising growing a genetically modified microbe under nutrient starvation condition, wherein the genetically modified microbe comprises a deletion of one or more nucleotides of at least one endogenous nucleic acid encoding an acyl (fatty acid) CoA ligase, and wherein the genetically modified microbe displays an accumulation of medium chain length 3-hydroxyacyl acids; and recovering medium chain length 3-hydroxyacyl acids produced by the microbe.

In some embodiments, the 3-hydroxyacyl acids are secreted from the cells and accumulated in the culture supernatant. The amount of 3-hydroxyacyl acids accumulation in the culture supernatant can be measured based on any suitable analytic methods. In some embodiments, the medium chain length 3-hydroxyacyl acids are accumulated at a titer of at least 0.2 g/L. In some embodiments, the medium chain length 3-hydroxyacyl acids are accumulated at a titer of at least 0.5 g/L. In some embodiments, the medium chain length 3-hydroxyacyl acids are accumulated at a titer greater than 1 g/L. In some embodiments, the medium chain length 3-hydroxyacyl acids are accumulated at a titer of at least 1.2 g/L. In some embodiments, the medium chain length 3-hydroxyacyl acids are accumulated at a titer of at least 1.5 g/L. In some embodiments, the medium chain length 3-hydroxyacyl acids are accumulated at a titer of at least 2.0 g/L. In some embodiments, the medium chain length 3-hydroxyacyl acids are accumulated at a titer of at least 2.5 g/L. In some embodiments, the medium chain length 3-hydroxyacyl acids are accumulated at a titer of at least 3.0 g/L.

In some embodiments, the amount of 3-hydroxyacyl acids accumulation in the culture supernatant is measured by taking samples, centrifuging to remove the cells, acidifying the supernatant with sulfuric acid, and then running on HPLC.

The medium chain length 3-hydroxyacyl acids accumulated in the culture supernatant can be recovered by any suitable techniques. In some embodiments, the medium chain length 3-hydroxyacyl acids are purified using solid-phase extraction.

The specific examples listed below are only illustrative and by no means limiting.

### EXAMPLES

### Example 1: Identification of CoA Ligases to Delete which would Result in Accumulation of 3-Hydroxyacids in the Culture Supernatant

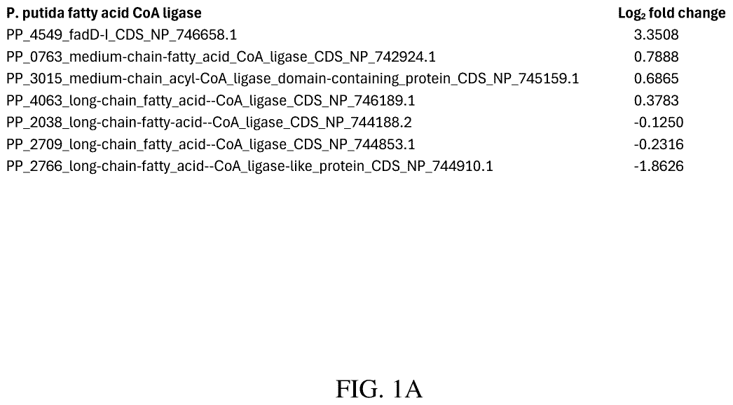

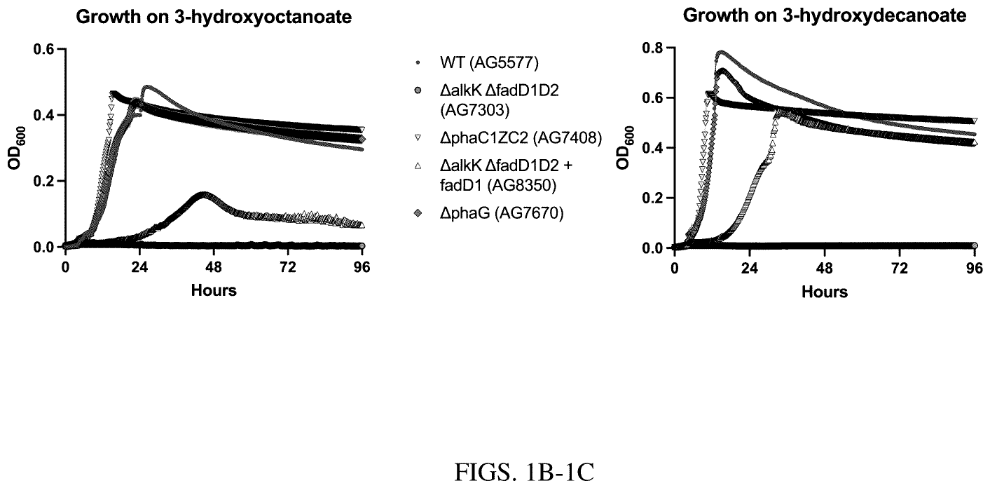

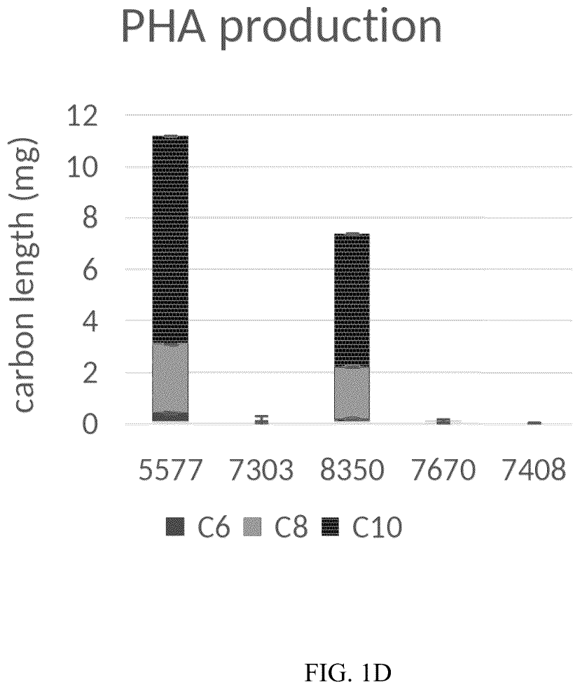

It was hypothesized that PhaG is a thiolase, then deletion of P. putida native 3-hydroxyacid:CoA ligases would result in accumulation of 3-hydroxyacids, a valuable industrial chemical, in the culture supernatant. It was hypothesized that PhaG generates 3-hydroxyacids. Therefore, it was sought to identify CoA ligases that would activate the resulting free 3-hydroxyacyl acid into the 3-hydroxyacyl-CoA that is needed for PHA polymerization. To find CoA ligase(s) responsible for this conversion, RNAseq analysis was performed to identify differentially expressed genes when P. putida KT2440 was grown on 3-hydroxydecanoate or acetate as the sole carbon source (FIG. 1A). fadD1 (PP_4549) and alkK (PP_0763) were the most and second most upregulated genes during growth on 3-hydroxydecanoate, respectively (FIG. 1A).

### Example 2: Lack of CoA Ligase Activity would Result in Production of 3-Hydroxyacyl Acids

Based on the results as described above, the genes encoding FadD1 and AlkK were deleted. The resulting strain AG7303 could no longer grow on 3-hydroxyoctanoate or 3-hydroxydecanoate (FIGS. 1B-1C), suggesting that these genes are responsible for most or all medium chain length 3-hydroxyacid:CoA ligase activity in the cell.

As 3-hydroxydecanoyl (C10) and 3-hydroxyoctanoyl (C8) units comprise the majority mcl-PHA building blocks, it was sought to determine if PHA production was affected by these CoA ligase deletions. These mutant strains were each grown in nitrogen-limited medium containing 25 mM p-coumaric acid (p-CA) and 2 mm (NH₄)₂SO₄ in triplicate 50 mL shake flask cultures for 72 hours. Cell pellets were then collected and processed for GC/MS analysis. It was found that PHA production was largely abolished in the absence of fadD1 and alkK (i.e., in strain AG7303), and PHA production was recovered by the reintroduction of fadD1 in the double deletion (strain AG8350) (FIG. 1D). Strains lacking phaG (AG7670) or the PHA polymerases phaCIC2 (AG7408) also did not produce PHAs, as expected.

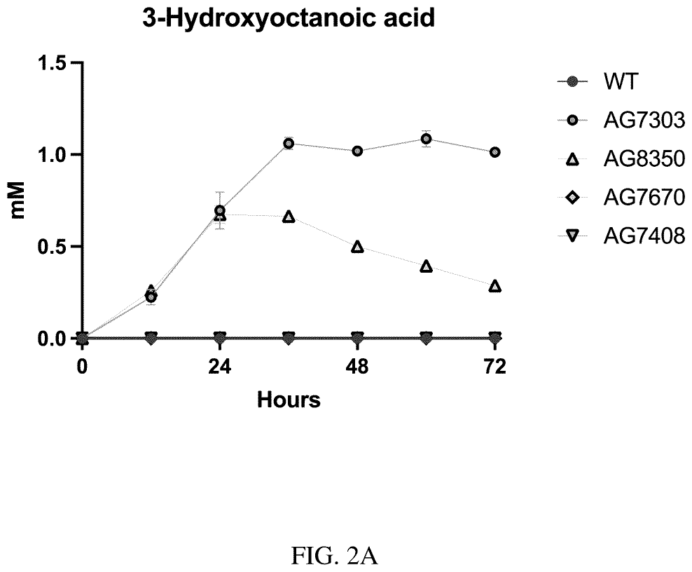

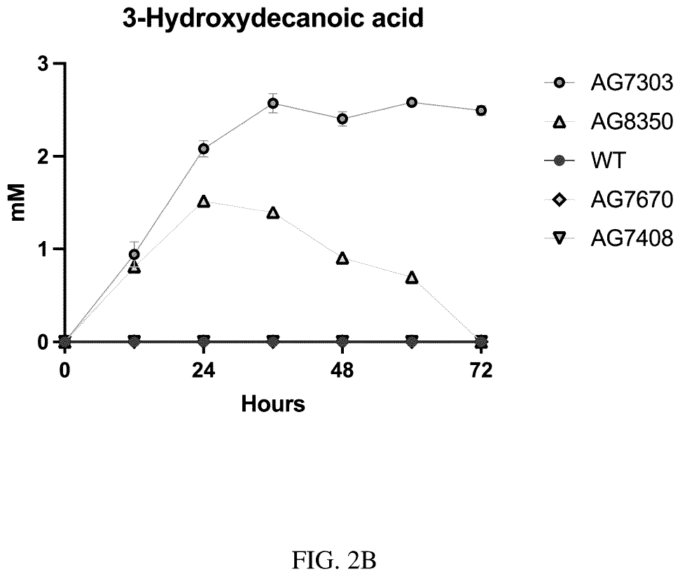

It was hypothesized that a lack of CoA ligase activity in the presence of PhaG would result in production of 3-hydroxyacyl acids. Therefore, the culture supernatant of P. putida ΔalkK ΔfadD1D2 was examined using HPLC (FIG. 2). This strain indeed accumulated 3-hydroxyoctanoate and 3-hydroxydecanoate in the supernatant when grown on p-coumarate (FIGS. 2A and 2B) and a variety of other carbon substrates (not shown), under nitrogen starvation conditions. Control strains lacking PhaG or the PHA polymerase PhaZ did not accumulate 3-hydroxyacyl acids, indicating this is not a general response to disrupted PHA production. Overall, the present disclosure demonstrates that PhaG is a 3-hydroxyacyl-ACP thiolase and that deletion of the corresponding CoA ligases is a promising route for the commercial production of medium chain length 3-hydroxyacyl acids such as 3-hydroxyoctanoate and 3-hydroxydecanoate.

The results as disclosed above strongly suggest that PhaG bears a thioesterase activity especially considering the necessity of a medium-chain CoA ligase for P. putida to produce PHAs (Wang et al, Appl Environ Microbiol. 2012 January; 78 (2): 519-27). The surprising production and accumulation of mcl 3-hydroxyacids by P. putida from low value carbon sources, when the CoA ligases AlkK and FadD are deleted, promises to be economically and environmentally valuable.

### Example 3: Materials and Methods

### Strain Cultivation

Cultures of E. coli and P. putida were grown at 37° C. and 30° C., respectively, in LB (Miller) broth (10 g/L tryptone, 5 g/L yeast extract, and 10 g/L NaCl) or on agar during routine culturing and preparation of competent cells. Antibiotics for plasmid selection and maintenance were used as required: 50 μg/mL kanamycin, 50 μg/mL apramycin, and 100 μg/mL ampicillin. For maintenance of temperature sensitive plasmids, cultures were incubated at 22° C.

### Plasmid Construction

Phusion® or Q5® High-Fidelity Polymerases (NEB) were used for all PCR amplifications and DpnI (NEB) digestion was used to remove plasmid templates from PCR amplified products. NEBuilder® HiFi DNA Assembly Master Mix (NEB) was used for plasmid construction. The plasmids were propagated using NEB 5-α F′I^{q} E. coli grown in LB Miller supplemented with requisite antibiotics for plasmid selection and maintenance. Plasmids were extracted from overnight cultures using the geneJET Plasmid Miniprep Kit (Thermo Fisher Scientific). The plasmids and oligonucleotides used for this study are listed in Table 1 and 2, respectively. Oligonucleotides were purchased from Eurofins Genomics (Louisville, KY, USA).

### RNAseq Analysis

P. putida strain AG5577 was cultured aerobically in flasks shaking at 230 rpm at 30° C. with the sole carbon source being 25 mM acetate or 5 mM 3-hydroxydecanoate from a starting OD₆₀₀ of 0.1 until reaching OD₆₀₀ of 0.3. The cultures were centrifuged to generate cell pellets and RNA was extracted and sequenced by CD Genomics. The relative abundance of mRNA for each CoA ligase was calculated and ranked from those most upregulated when the culture was grown on 3-hydroxydecanoate. A greater than zero log₂ fold change indicates an increase in abundance and less than zero indicates a decrease. CoA ligases upregulated in the 3-hydroxydecanoate treatment were identified for gene deletion.

P. putida Strain Construction

Genes were deleted using pK18mobsacB-derived plasmids using the previously described kanamycin selection and sucrose counter selection method (Marx, BMC Res Notes 1, 1 (2008); Johnson et al, Metab. Eng. 2015, 28:240-247). fadD1 complementation was achieved with BxB1 and ΦC31 serine integrase mediated genome integration and plasmid backbone excision (Elmore Sci. Adv., 2023). Electrocompetent P. putida cells were prepared by centrifuging 50 mL of an overnight culture, started from glycerol stock, at 5000 rcf at 22° C. and washing the cell pellet in 10% glycerol three times, followed by a final resuspension in 1 mL 10% glycerol. 300-800 ng of gene deletion plasmids or 20 ng of integrase and BxB1 cargo plasmids were mixed with 50 μL of competent cells and electroporated in 1 mm gap cuvettes using a Gene Pulser Xcell (Bio-Rad) set at 1600 V, 25 μF, 2002.

### Batch Fermentations and HPLC Analysis of Hydroxy Acid Production

Starter cultures were grown overnight from single colonies and then used to inoculate fresh media with a 1% inoculum in shake flasks. M9 minimal media was used with various single carbon sources and low ammonium sulfate concentrations to induce PHA/hydroxy acid production. The carbon sources included cellulosic sugars (glucose, xylose, arabinose), lignin-related aromatics (p-coumarate, ferulate), deconstructed plastics (terephthalate), organic acids (acetate), and glycerol. These were grown at 30° C. shaking at 200-250 rpm for up to 96 hours with 500 μL samples periodically collected for HPLC analysis. After centrifugation the samples were acidified with H₂SO₄, filtered through 0.22 micron spin filter and 5 μL were loaded to a 1260 Infinity II HPLC (Agilent Technologies), using an autosampler, running an isocratic 5 mM H₂SO₄ solvent at 0.6 mL/minute and separated on a Fast-Acid column (Bio-Rad) heated to 60° C. and quantified with a refractive index detector (RID) maintained at 35° C. Standard curves were generated using Agilent OpenLab CDS for 3-hydroxyhexanoate, 3-hydroxyoctanoate, and 3-hydroxydecanoate (Sigma) as well as each carbon source.

### MS/GC Method for PHA Derivatization and Analysis

Triplicate 5 mL cultures were grown overnight in M9 media with 25 mM p-coumarate as sole carbon source and a growth limiting 4 mM of NH₄. 500 μL of these overnight cultures were used to inoculate 50 mL of the same M9 media in 250 Erlenmeyer flasks that were then incubated at 30° C. shaking at 250 rpm. After 72 hours, the cultures were centrifuged, and the cell pellets were washed twice with H₂O and lyophilized. Samples were prepared for derivatization by adding 10-30 mg of lyophilized biomass to a GC vial. To track derivatization, 200 μL of benzoic acid dissolved in dichloromethane (10 mg/mL) was added as an internal surrogate. Samples were derivatized by adding ˜1 mL of BF₃/MeOH to the GC vial, which was sealed, shaken, and placed in a heating block at 80° C. overnight. Vials were then removed from the heating block and allowed to cool to room temperature. Vial contents were pipetted into a 10 mL volumetric flask and the vial residual was rinsed twice with dichloromethane (DCM) before filling the flask to 10 mL total with additional DCM. The 10 mL solution was transferred to a PTFE capped vial and ˜3 mL of water was added to form a bi-phase and wash out residual BF₃ to the aqueous layer. The DCM layer (˜2 mL) was then transferred into another gram vial containing Na₂SO₄ and Na₂CO₃ to dry and neutralize any remaining BF₃. The dried and neutralized solutions were syringe filtered (0.2-μm PTFE) into fresh GC vials for analysis.

TABLE 1
Plasmids used in the study

| Plasmid | Utility | Construction details |
|---|---|---|
| pWTW009 | Deletion of P. putida | Linearized pk18mobsacB plasmid (oWWTW0010 & |
| | fadD1D2 (PP_4549- | oWTW0011) assembled with homology arms from upstream |
| | 4550) | (oWTW0038 & oWTW0039) and downstream (oWTW0036 & |
| | | oWTW0037) of fadD1D2 using Gibson assembly. |
| pWTW012 | Complementation of | Bxb1 serine integrase cargo plasmid containing PP_4549 |
| | P. putida fadD1 | (fadD1) with 301 bp of the upstream region amplified from |
| | (PP_4549) | KT2440 gDNA (oWTW0113 & oWTW0115) and inserted into |
| | | PCR linearized pWTW002 (oWTW0110 & oWTW0114) using |
| | | Gibson assembly |
| pJH299 | Deletion of P. putida | EcoRI and HindIII linearized pk18mobsacB assembled with |
| | alkK (PP_0763) | homology arms from upstream (oJH0722 & oJH0723) and |
| | | downstream (oJH0724 & oJH0725) of alkK using Gibson |
| | | assembly. |
| pJH316 | Deletion of P. putida | Synthesized by Genscript. |
| | phaG (PP_1408) | |
| pJE473 | Deletion of P. putida | Elmore et al, Nat Commun, 12, 2261 (2021) |
| | phaC1ZC2 | |

TABLE 2
DNA Sequences of oligonucleotides used in the study.
Integrated DNA Technologies and Eurofins were used for oligonucleotide
synthesis. Plasmidsaurus was used for whole plasmid sequencing.

| Primer | Sequence (5′a3′) |
|---|---|
| oWTW0010 | TGGCACTGGCCGTCGTTT |
| oWTW0011 | CGTAATCATGTCATAGCTGTTTCCTGAG |
| oWTW0036 | ACAGCTATGACATGATTACGCCATGGGCGGTATCGTCAC |
| oWTW0037 | ACAATAATAACCACAAGCCTGCACGCTC |
| oWTW0038 | AGGCTTGTGGTTATTATTGTTCCTCTGCCTGGACCTGC |
| oWTW0039 | TAAAACGACGGCCAGTGCCACGGTACAGCACGGCCGGT |
| oWTW0110 | GATCGCCTGAAACGCATGAGAAAGCCCC |
| oWTW0113 | CTCATGCGTTTCAGGCGATCTTCTTCAAG |
| oWTW0114 | GTGTAGGAGCCGGACCAAAACGAAAAAAGG |
| oWTW0115 | TTTTGGTCCGGCTCCTACACTGCCCAATG |
| oJH0722 | TCACTCAGGAAACAGCTATGACATGATTACGCATACCGAAGGCTTCGGCC |
| | AGCCT |
| oJH0723 | GGATCCGTAACGTACTCTAGAACGGCCAAATCAGCCGAA |
| oJH0724 | TCTAGAGTACGTTACGGATCCTAACCATTGTGCGGGTCGCGC |
| oJH0725 | GTCACGACGTTGTAAAACGACGGCCAGTGCCACGACTTGGCGCCTTCCTT |

TABLE 3
Strains and construction details for bacterial strains used in the study.

| Strain | Genotype | Details |
|---|---|---|
| AG5577 | P. putida KT2440 | Otherwise wild type strain with three SAGE |
| | ΔPP_2876::R4_phiBT1_MR11_attB | landing pads (Elmore 2023) |
| | cassette | |
| | ΔPP_4740::BxBI_RV_phi370_attB | |
| | cassette ΔPP_4217/4218 | |
| | intergenic::TG1_BL3_A118_attB cassette | |
| AG7303 | P. putida KT2440 | P. putida AG5577 transformed with pJH299 |
| | ΔPP_2876::R4_phiBT1_MR11_attB | and pWTW009 |
| | cassette | |
| | ΔPP_4740::BxBI_RV_phi370_attB | |
| | cassette ΔPP_4217/4218 | |
| | intergenic::TG1_BL3_A118_attB cassette | |
| | ΔalkK ΔfadD1D2 | |
| AG7408 | P. putida KT2440 | P. putida AG5577 transformed with pJE473 |
| | ΔPP_2876::R4_phiBT1_MR11_attB | |
| | cassette | |
| | ΔPP_4740::BxBI_RV_phi370_attB | |
| | cassette ΔPP_4217/4218 | |
| | intergenic::TG1_BL3_A118_attB cassette | |
| | ΔphaC1C2 | |
| AG7670 | P. putida KT2440 | P. putida AG5577 transformed with pJH316 |
| | ΔPP_2876::R4_phiBT1_MR11_attB | |
| | cassette ΔPP_4740::BxBI | |
| | attL-_RV_phi370_attB cassette ΔPP_4217/4218 | |
| | intergenic::TG1_BL3_A118_attB cassette | |
| | ΔphaG | |
| AG7790 | P. putida KT2440 ΔhsdR::Bxb1int-attB | P. putida AG3916 transformed with pJH299 |
| | Δgcd::araE-araCDABE ΔampC::xylE- | and pWTW009. (This strain can make |
| | xylAB_talB_tktA(imp. xylE promoter) Δcrc | hydroxy acids from arabinose and xylose) |
| | ΔalkK ΔfadD1D2 | |
| AG8350 | P. putida KT2440 | P. putida AG7303 transformed with |
| | ΔPP_2876::R4_phiBT1_MR11_attB | pWTW012 |
| | cassette ΔPP_4740::BxBIattL-fadD1- | |
| | attR_RV_phi370_attB cassette | |
| | ΔPP_4217/4218 | |
| | intergenic::TG1_BL3_A118_attB cassette | |
| | ΔalkK ΔfadD1D2 | |
| AG8974 | P. putida KT2440::Ptac-tph2::Ptac-tpaK. | P. putida AG5475 transformed with pJH299 |
| | ΔalkK ΔfadD1D2 | and pWTW009 |
| | | (This strain can make hydroxy acids from |
| | | terephthalic acid) |

What is claimed is:

1. A genetically modified microbe, comprising a deletion of one or more nucleotides of at least one endogenous nucleic acid encoding an acyl (fatty acid) CoA ligase, wherein the genetically modified microbe displays an accumulation of medium chain length 3-hydroxyacyl acids under a nutrient starvation condition.

2. The genetically modified microbe of claim 1, wherein the acyl CoA ligase is a medium chain length acyl CoA ligase.

3. The genetically modified microbe of claim 2, wherein the acyl CoA ligase is a 3-hydroxy acyl-CoA ligase.

4. The genetically modified microbe of claim 1, wherein the acyl CoA ligase is selected from FadD1 or a homolog thereof, FadD2 or a homolog thereof, or AlkK or a homolog thereof.

5. The genetically modified microbe of claim 4, wherein the acyl CoA ligase is FadD1 or a homolog thereof.

6. The genetically modified microbe of claim 1, wherein the at least one endogenous nucleic acid comprises multiple endogenous nucleic acids, each encoding an acyl CoA ligase.

7. The genetically modified microbe of claim 6, wherein the at least one endogenous nucleic acid comprises an endogenous nucleic acid encoding FadD1 or a homolog thereof, and an endogenous nucleic acid encoding AlkK or a homolog thereof.

8. The genetically modified microbe of claim 4, wherein the nucleic acid encoding the FadD1 or a homolog thereof comprises a nucleotide sequence having at least 90% sequence identity to the nucleotide sequence shown in SEQ ID NO: 1.

9. The genetically modified microbe of claim 4, wherein the FadD1 or the homolog thereof comprises an amino acid sequence having at least 90% sequence identity to the amino acid sequence shown in SEQ ID NO: 2.

10. The genetically modified microbe of claim 4, wherein the nucleic acid encoding the AlkK or a homolog thereof comprises a nucleotide sequence having at least 90% sequence identity to the nucleotide sequence shown in SEQ ID NO: 3.

11. The genetically modified microbe of claim 4, wherein the AlkK or the homolog thereof comprises an amino acid sequence having at least 90% sequence identity to the amino acid sequence shown in SEQ ID NO: 4.

12. The genetically modified microbe of claim 4, wherein the nucleic acid encoding the FadD2 or a homolog thereof comprises a nucleotide sequence having at least 90% sequence identity to the nucleotide sequence shown in SEQ ID NO: 5.

13. The genetically modified microbe of claim 4, wherein the FadD2 or the homolog thereof comprises an amino acid sequence having at least 90% sequence identity to the amino acid sequence shown in SEQ ID NO: 6.

14. The genetically modified microbe of claim 1, wherein the deletion of one or more nucleotides of at least one endogenous nucleic acid encoding an acyl CoA ligase is a deletion of a portion of the at least one endogenous nucleic acid, and the resulting nucleic acid does not encode a functional acyl CoA ligase.

15. The genetically modified microbe of claim 1, wherein the deletion of one or more nucleotides of at least one endogenous nucleic acid encoding an acyl CoA ligase is a deletion of the full length of the at least one endogenous nucleic acid.

16. The genetically modified microbe of claim 1, wherein the microbe is Pseudomonas.

17. The genetically modified microbe of claim 16, wherein the microbe is Pseudomonas putida.

18. The genetically modified microbe of claim 1, wherein the medium chain length 3-hydroxyacyl acids comprises 6-12 carbon chain length compounds.

19. The genetically modified microbe of claim 1, wherein the nutrient starvation condition comprises nitrogen starvation condition.

20. The genetically modified microbe of claim 19, wherein the genetically modified microbe is grown on an organic compound.

21. The genetically modified microbe of claim 20, wherein the organic compound comprises a carbon source.

22. The genetically modified microbe of claim 21, wherein the carbon source comprises glucose, arabinose, xylose, glycerol, benzoate, acetate, p-coumaric acid, or terephthalate.

23. A method of producing medium chain length 3-hydroxyacyl acids comprising
growing a genetically modified microbe under nutrient starvation condition, wherein the genetically modified microbe comprises a deletion of one or more nucleotides of at least one endogenous nucleic acid encoding an acyl (fatty acid) CoA ligase, and wherein the genetically modified microbe displays an accumulation of medium chain length 3-hydroxyacyl acids; and
recovering medium chain length 3-hydroxyacyl acids produced by the microbe.

24. The method of claim 23, wherein the acyl CoA ligase is a medium chain length acyl CoA ligase.

25. The method of claim 24, wherein the acyl CoA ligase is a 3-hydroxy acyl-CoA ligase.

26. The method of claim 23, wherein the acyl CoA ligase is selected from FadD1 or a homolog thereof, FadD2 or a homolog thereof, or AlkK or a homolog thereof.

27. The method of claim 26, wherein the acyl CoA ligase is FadD1 or a homolog thereof.

28. The method of claim 23, wherein the at least one endogenous nucleic acid comprises multiple endogenous nucleic acids, each encoding an acyl CoA ligase.

29. The method of claim 28, wherein the at least one endogenous nucleic acid comprises an endogenous nucleic acid encoding FadD1 or a homolog thereof, and an endogenous nucleic acid encoding AlkK or a homolog thereof.

30. The method of claim 26, wherein the nucleic acid encoding the FadD1, or a homolog thereof comprises a nucleotide sequence having at least 90% sequence identity to the nucleotide sequence shown in SEQ ID NO: 1.

31. The method of claim 26, wherein the FadD1 or the homolog thereof comprises an amino acid sequence having at least 90% sequence identity to the amino acid sequence shown in SEQ ID NO: 2.

32. The method of claim 26, wherein the nucleic acid encoding the AlkK or a homolog thereof comprises a nucleotide sequence having at least 90% sequence identity to the nucleotide sequence shown in SEQ ID NO: 3.

33. The method of claim 26, wherein the AlkK or the homolog thereof comprises an amino acid sequence having at least 90% sequence identity to the amino acid sequence shown in SEQ ID NO: 4.

34. The method of claim 26, wherein the nucleic acid encoding the FadD2, or a homolog thereof comprises a nucleotide sequence having at least 90% sequence identity to the nucleotide sequence shown in SEQ ID NO: 5.

35. The method of claim 26, wherein the FadD2 or the homolog thereof comprises an amino acid sequence having at least 90% sequence identity to the amino acid sequence shown in SEQ ID NO: 6.

36. The method of claim 23, wherein the deletion of one or more nucleotides of at least one endogenous nucleic acid encoding an acyl CoA ligase is a deletion of a portion of the at least one endogenous nucleic acid, and the resulting nucleic acid does not encode a functional medium chain fatty acid CoA ligase.

37. The method of claim 23, wherein the deletion of one or more nucleotides of at least one endogenous nucleic acid encoding an acyl CoA ligase is a deletion of the full length of the at least one endogenous nucleic acid.

38. The method of claim 23, wherein the microbe is Pseudomonas.

39. The method of claim 38, wherein the microbe is Pseudomonas putida.

40. The method of claim 23, wherein the medium chain length 3-hydroxyacyl acids comprises 6-12 carbon chain length compounds.

41. The method of claim 23, wherein the nutrient starvation condition comprises nitrogen starvation condition.

42. The method of claim 41, wherein the genetically modified microbe is grown on an organic compound.

43. The method of claim 42, wherein the organic compound comprises a carbon source.

44. The method of claim 43, wherein the carbon source comprises glucose, arabinose, xylose, glycerol, benzoate, acetate, p-coumaric acid, or terephthalate.


SEQUENCE LISTING (20 sequences)

SEQ ID NO: 1 - DNA, 1698 bp, Pseudomonas putida
  source 1..1698: mol_type = other DNA
atgatcgaaaatttttggaaggataagtacccagccgggattacggcggaaatcaatcctgacgaattccccaatatccaggcagtactcaagcaatcctgccaacgctttgccgacaaaccggcctttagcaacctgggcaagactatcacttatggcgagttgtatgcgttgtcgggggcgtttgccgcctggctgcagcagcataccgacctcaagccgggtgaccgcattgccgtgcaactgcccaatgtcctgcaatacccggtcgcggtcttcggtgccatgcgtgccgggctgatcgtggtcaacaccaacccgctgtacaccgcgcgggagatggaacaccagttcaacgactcgggtgccaaggccctggtgtgcctggccaacatggcccacctggcggaaaaagtggtgcccaaaacccaggtcaggcacgttatcgtcactgaagttgccgacctgctgccaccactcaagcgcctgctgatcaacagcgtgatcaagtacgtgaagaagatggtgccggcctacaacctgccgcaggccgtgcgtttcaatgacgccctggcgctgggcaagggccagcccgtgaccgaagccaacccgcaggccaacgacgtggcggtgctgcagtacaccggcggtaccaccggtgtggccaaaggcgccatgctgacccaccgcaacctggtggccaacatgctgcagtgccgtgcactgatgggctccaacctgcacgaaggctgcgaaatcctcatcaccccgctgccgctgtaccacatctatgcgtttaccttccattgcatggcgatgatgctgatcggcaaccacaacgtgctgatcagcaacccgcgtgacttgccggcgatggtcaaggaactgggcaagtggaagttcagcggctttgtgggcctcaacaccctgttcgttgccctgtgcaacaacgaggcgttccgagccctggatttctcggcgctgaaaatcaccctgtcgggcggtatggccttgcagctaagcgtggccgagcgctggaaggccgttaccggttgcgccatctgcgaaggctacggcatgaccgaaaccagcccggtggcggcggtgaacccctcggaagcgaaccaggtgggcaccatcggtattccggtgccgtcgaccctgtgcaaggtcatcgacgacgccggcaatgagttgccgcttggcgaagtgggcgagctgtgtgtcaagggcccgcaggtgatgaagggctactggcagcgtgaagacgccacggccgagattctcgacagcgaaggctggctgaagaccggcgacatcgccgtgatccaggcggacggctacatgcgcatcgtcgaccgcaagaaagacatgatcctggtctcgggcttcaacgtataccccaacgagctggaagacgtgttggcggccctgccgggcgtgctgcagtgcgcagccatcggtgtgccggacgagaagtcgggtgaagtgatcaaggtcttcatcgtggtcaagccgggcatgaccgtgaccaaggagcaggtgatggagcacatgcgtgccaacgttaccggctacaaggtaccgcgccacatcgagttccgcgattcgctgccgaccaccaacgtgggcaagatcctgcgccgcgaactgcgtgatgaagagctcaagaagcaaggcttgaagaagatcgcctga

SEQ ID NO: 2 - protein, 565 aa, Pseudomonas putida
MIENFWKDKYPAGITAEINPDEFPNIQAVLKQSCQRFADKPAFSNLGKTITYGELYALSGAFAAWLQQHTDLKPGDRIAVQLPNVLQYPVAVFGAMRAGLIVVNTNPLYTAREMEHQFNDSGAKALVCLANMAHLAEKVVPKTQVRHVIVTEVADLLPPLKRLLINSVIKYVKKMVPAYNLPQAVRFNDALALGKGQPVTEANPQANDVAVLQYTGGTTGVAKGAMLTHRNLVANMLQCRALMGSNLHEGCEILITPLPLYHIYAFTFHCMAMMLIGNHNVLISNPRDLPAMVKELGKWKFSGFVGLNTLFVALCNNEAFRALDFSALKITLSGGMALQLSVAERWKAVTGCAICEGYGMTETSPVAAVNPSEANQVGTIGIPVPSTLCKVIDDAGNELPLGEVGELCVKGPQVMKGYWQREDATAEILDSEGWLKTGDIAVIQADGYMRIVDRKKDMILVSGFNVYPNELEDVLAALPGVLQCAAIGVPDEKSGEVIKVFIVVKPGMTVTKEQVMEHMRANVTGYKVPRHIEFRDSLPTTNVGKILRRELRDEELKKQGLKKIA

SEQ ID NO: 3 - DNA, 1683 bp, Pseudomonas putida
  source 1..1683: mol_type = other DNA
atgttgcagacacgcatcatcaagcccgccgagggcgcctatgcctatccattgctgatcaagcgcctgctgatgtccggcagccgctatgaaaagacccgggaaatcgtctaccgcgaccagatgcggctgacgtatccacagctcaacgagcgcattgcccgcctggccaacgtgctgaccgaggccggggtcaaggccggtgacaccgtggcggtgatggactgggacagccatcgctacctggaatgcatgttcgccatcccgatgatcggcgctgtggtgcacaccatcaacgtgcgcctgtcgcccgagcagatcctctacaccatgaaccatgccgaagaccgcgtggtgctggtcaacagcgacttcgtcggcctgtaccaggccatcgccgggcagctgaccactgtcgacaagaccctgctactgaccgatggcccggacaagactgccgaactgcccggtctggtcggcgagtatgagcagctgctggctgctgccagcccgcgctacgacttcccggatttcgacgagaattcggtggccactaccttctacaccactggcaccaccggtaaccccaagggcgtgtatttcagtcaccgccagctggtgctgcacaccctggccgaggcctcggtcaccggcagtatcgacagcgtgcgcctgctgggcagcaacgatgtgtacatgcccatcaccccgatgttccacgtgcatgcctggggcatcccctacgctgccaccatgctcggcatgaagcaggtgtacccagggcgctacgagccggacatgctggtcaagctttggcgtgaagagaaggtcactttctcccactgcgtgccgaccatcctgcagatgctgctcaactgcccgaacgcccaggggcaggacttcggcggctggaagatcatcatcggcggcagctcgctcaaccgttcgctgtaccaggccgccctggcgcgcggcatccagctgaccgccgcgtatggcatgtcggaaacctgcccgctgatctccgcggcacacctgaacgatgaactgcaggccggcagcgaggatgagcgcgtcacttaccgtatcaaggccggtgtgccggtgccgttggtcgaagcggccatcgtcgacggcgaaggcaacttcctgcccgccgatggtgaaacccagggcgagctggtactgcgtgcgccgtggctgaccatgggctacttcaaggagccggagaagagcgaggagctgtggcagggcggctggctgcacaccggtgacgtcgccaccctcgacggcatgggctacatcgacatccgcgaccgcatcaaggatgtgatcaagaccggtggcgagtgggtttcctcgctcgacctggaagacctgatcagccgccacccggccgtgcgcgaagtggcggtggtgggggtggccgacccgcagtggggtgagcgcccgtttgccctgctggtggcacgtgacggccacgatatcgacgccaaggcgctgaaggaacacctcaagccattcgtcgagcaaggtcatatcaacaagtgggcgattccaagccagatcgcccttgttactgaaattcccaagaccagtgtcggcaagctcgacaagaaacgcattcgccaggacatcgtccagtggcaggccagcaacagcgcgttcctttccacgttgtaa

SEQ ID NO: 4 - protein, 560 aa, Pseudomonas putida
MLQTRIIKPAEGAYAYPLLIKRLLMSGSRYEKTREIVYRDQMRLTYPQLNERIARLANVLTEAGVKAGDTVAVMDWDSHRYLECMFAIPMIGAVVHTINVRLSPEQILYTMNHAEDRVVLVNSDFVGLYQAIAGQLTTVDKTLLLTDGPDKTAELPGLVGEYEQLLAAASPRYDFPDFDENSVATTFYTTGTTGNPKGVYFSHRQLVLHTLAEASVTGSIDSVRLLGSNDVYMPITPMFHVHAWGIPYAATMLGMKQVYPGRYEPDMLVKLWREEKVTFSHCVPTILQMLLNCPNAQGQDFGGWKIIIGGSSLNRSLYQAALARGIQLTAAYGMSETCPLISAAHLNDELQAGSEDERVTYRIKAGVPVPLVEAAIVDGEGNFLPADGETQGELVLRAPWLTMGYFKEPEKSEELWQGGWLHTGDVATLDGMGYIDIRDRIKDVIKTGGEWVSSLDLEDLISRHPAVREVAVVGVADPQWGERPFALLVARDGHDIDAKALKEHLKPFVEQGHINKWAIPSQIALVTEIPKTSVGKLDKKRIRQDIVQWQASNSAFLSTL

SEQ ID NO: 5 - DNA, 1689 bp, Pseudomonas putida
  source 1..1689: mol_type = other DNA
atgcaagccgacttctggaatgacaagcgcccggcaggcgtgccttccaccatcgacatcaatgcctacgcctcggtcgtcgaggtgttcgagcgctcctgcaagcgctttgccgaccgcccggcgttcagcaacctgggcgtgaccctcagctacgcggaactggagcgccattcggcagccttcgctgcctggttgcagcagcacaccgacctcaaaccgggtgagcgcatcgccgtacagatgcccaatgtgctgcagtaccccatcgccgtcttcggtgccatgcgcgccgggctgatcgtggtcaataccaacccgctgtacaccgagcgcgagatgcgccaccagttcaaggacagtggcgcgcgtgcgctggtgtacctgaacatgttcggcaagcgcgtgcaggaggtgctgcccgataccggtatcgaatacctgatcgaggcaaagatgggtgacctgctgccggccgccaagggctggctggtcaacaccgtggtcgacaagttgaagaagatggtgccggcctaccggttgccccaggcggtgccgttcaagcaggtgctgcgcgagggccgcgggctgtcgcccaaaccggtgtcgctgaacctcgatgacatcgcggtgctgcagtacaccggcggcaccaccggcctggccaagggcgccatgctcacccatggcaacctggtggccaacatgctgcaagtgctggcctgcttctcgcagcacggccccgatgggcagaagctgctgaaggacggccaggaagtgatgattgcgccgctgccgctgtaccacatctatgccttcaccgcgaactgcatgtgcatgatggtcaccggcaaccataatgtgctgattaccaacccgcgggatattcccggcttcatcaaggagctgggcaagtggcgtttctctgccttgctgggcctcaataccctgtttgtcgcactgatggaccacccgggattccgtcagctggacttctcggcgctgaaggtcaccaactctggtggcacagcgctggtcaaagccaccgccgagcgctgggaagaccttaccgggtgccgcatcgttgaaggctacggcctgacagaaacctcgccggtggccagcactaacccctacggccagctggcgcgtctgggtaccgtgggcatccccgtggcgggtaccgcgttcaaggtcatcgatgatgatggcaacgaactgccgctgggcgagcggggcgagctttgcatcaaaggcccgcaggtcatgaaggggtactggcagcagcccgaggcaacggcccaggcactggatgcagaaggctggttcaagaccggtgatatcgcggtgatcgacccggacggcttcacccgaattgtcgaccgcaagaaggacatgatcatcgtctcgggcttcaacgtgtaccccaacgaaatcgaggatgtggtgatggggcatcccaaggtcgccaactgtgcggctatcggcgtgccggacgagcgttccggcgaggcggtcaagctgttcgtggtgccgcgcgagggggggctgagcgttgatgagctgaaggcctactgcaaggccaacttcaccggctacaaagtacccaagcacatcgtgctgcgcgaatcgttgcccatgacgccggtgggcaagattttgcggcgggagttgcgcgatatagcgtga

SEQ ID NO: 6 - protein, 562 aa, Pseudomonas putida
MQADFWNDKRPAGVPSTIDINAYASVVEVFERSCKRFADRPAFSNLGVTLSYAELERHSAAFAAWLQQHTDLKPGERIAVQMPNVLQYPIAVFGAMRAGLIVVNTNPLYTEREMRHQFKDSGARALVYLNMFGKRVQEVLPDTGIEYLIEAKMGDLLPAAKGWLVNTVVDKLKKMVPAYRLPQAVPFKQVLREGRGLSPKPVSLNLDDIAVLQYTGGTTGLAKGAMLTHGNLVANMLQVLACFSQHGPDGQKLLKDGQEVMIAPLPLYHIYAFTANCMCMMVTGNHNVLITNPRDIPGFIKELGKWRFSALLGLNTLFVALMDHPGFRQLDFSALKVTNSGGTALVKATAERWEDLTGCRIVEGYGLTETSPVASTNPYGQLARLGTVGIPVAGTAFKVIDDDGNELPLGERGELCIKGPQVMKGYWQQPEATAQALDAEGWFKTGDIAVIDPDGFTRIVDRKKDMIIVSGFNVYPNEIEDVVMGHPKVANCAAIGVPDERSGEAVKLFVVPREGGLSVDELKAYCKANFTGYKVPKHIVLRESLPMTPVGKILRRELRDIA

SEQ ID NO: 7 - DNA, 18 bp, synthetic construct
  misc_feature 1..18: note = Primer
  source 1..18: mol_type = other DNA
tggcactggccgtcgttt

SEQ ID NO: 8 - DNA, 28 bp, synthetic construct
  misc_feature 1..28: note = Primer
  source 1..28: mol_type = other DNA
cgtaatcatgtcatagctgtttcctgag

SEQ ID NO: 9 - DNA, 39 bp, synthetic construct
  misc_feature 1..39: note = Primer
  source 1..39: mol_type = other DNA
acagctatgacatgattacgccatgggcggtatcgtcac

SEQ ID NO: 10 - DNA, 28 bp, synthetic construct
  misc_feature 1..28: note = Primer
  source 1..28: mol_type = other DNA
acaataataaccacaagcctgcacgctc

SEQ ID NO: 11 - DNA, 38 bp, synthetic construct
  misc_feature 1..38: note = Primer
  source 1..38: mol_type = other DNA
aggcttgtggttattattgttcctctgcctggacctgc

SEQ ID NO: 12 - DNA, 38 bp, synthetic construct
  misc_feature 1..38: note = Primer
  source 1..38: mol_type = other DNA
taaaacgacggccagtgccacggtacagcacggccggt

SEQ ID NO: 13 - DNA, 28 bp, synthetic construct
  misc_feature 1..28: note = Primer
  source 1..28: mol_type = other DNA
gatcgcctgaaacgcatgagaaagcccc

SEQ ID NO: 14 - DNA, 29 bp, synthetic construct
  misc_feature 1..29: note = Primer
  source 1..29: mol_type = other DNA
ctcatgcgtttcaggcgatcttcttcaag

SEQ ID NO: 15 - DNA, 30 bp, synthetic construct
  misc_feature 1..30: note = Primer
  source 1..30: mol_type = other DNA
gtgtaggagccggaccaaaacgaaaaaagg

SEQ ID NO: 16 - DNA, 29 bp, synthetic construct
  misc_feature 1..29: note = Primer
  source 1..29: mol_type = other DNA
ttttggtccggctcctacactgcccaatg

SEQ ID NO: 17 - DNA, 55 bp, synthetic construct
  misc_feature 1..55: note = Primer
  source 1..55: mol_type = other DNA
tcactcaggaaacagctatgacatgattacgcataccgaaggcttcggccagcct

SEQ ID NO: 18 - DNA, 39 bp, synthetic construct
  misc_feature 1..39: note = Primer
  source 1..39: mol_type = other DNA
ggatccgtaacgtactctagaacggccaaatcagccgaa

SEQ ID NO: 19 - DNA, 42 bp, synthetic construct
  misc_feature 1..42: note = Primer
  source 1..42: mol_type = other DNA
tctagagtacgttacggatcctaaccattgtgcgggtcgcgc

SEQ ID NO: 20 - DNA, 50 bp, synthetic construct
  misc_feature 1..50: note = Primer
  source 1..50: mol_type = other DNA
gtcacgacgttgtaaaacgacggccagtgccacgacttggcgccttcctt